SEQUENCE LISTING (21 sequences)

SEQ ID NO: 1 - protein, 309 aa, Bacteriophage phi-29
MARKRSNTYRSINEIQRQKRNRWFIHYLNYLQSLAYQLFEWENLPPTINPSFLEKSIHQFGYVGFYKDPVISYIACNGALSGQRDVYNQATVFRAASPVYQKEFKLYNYRDMKEEDMGVVIYNNDMAFPTTPTLELFAAELAELKEIISVNQNAQKTPVLIRANDNNQLSLKQVYNQYEGNAPVIFAHEALDSDSIEVFKTDAPYVVDKLNAQKNAVWNEMMTFLGIKNANLEKKERMVTDEVSSNDEQIESSGTVFLKSREEACEKINELYGLNVKVKFRYDIVEQMRRELQQIENVSRGTSDGETNE

SEQ ID NO: 2 - protein, 4 aa, Artificial Sequence
  Linker
GGGS

SEQ ID NO: 3 - protein, 4 aa, Artificial Sequence
  Linker
PGGS

SEQ ID NO: 4 - protein, 4 aa, Artificial Sequence
  Linker
PGGG

SEQ ID NO: 5 - protein, 6 aa, Artificial Sequence
  Linker
RPPPPP

SEQ ID NO: 6 - protein, 5 aa, Artificial Sequence
  Linker
RPPPP

SEQ ID NO: 7 - protein, 4 aa, Artificial Sequence
  Linker
RPPG

SEQ ID NO: 8 - protein, 4 aa, Artificial Sequence
  Linker
PPPP

SEQ ID NO: 9 - protein, 4 aa, Artificial Sequence
  Linker
RPPG

SEQ ID NO: 10 - protein, 9 aa, Artificial Sequence
  Linker
PPPPPPPPP

SEQ ID NO: 11 - protein, 4 aa, Artificial Sequence
  Linker
GGGG

SEQ ID NO: 12 - protein, 5 aa, Artificial Sequence
  Linker
GGGGG

SEQ ID NO: 13 - protein, 6 aa, Artificial Sequence
  Linker
GGGGGG

SEQ ID NO: 14 - protein, 7 aa, Artificial Sequence
  Linker
DYDIPTT

SEQ ID NO: 15 - protein, 8 aa, Artificial Sequence
  Tag
WSHPQSEK

SEQ ID NO: 16 - protein, 8 aa, Artificial Sequence
  Tag
WSHPQFEK

SEQ ID NO: 17 - protein, 9 aa, Artificial Sequence
  Tag
NWSHPQFEK

SEQ ID NO: 18 - protein, 9 aa, Artificial Sequence
  Tag
PWSHPQFEK

SEQ ID NO: 19 - protein, 9 aa, Artificial Sequence
  Tag
GGSHPQFEG

SEQ ID NO: 20 - protein, 6 aa, Artificial Sequence
  Tag
HHHHHH

SEQ ID NO: 21 - protein, 7 aa, Artificial Sequence
  Cleavage site
ENLYFQG

(19) United States
(12) Patent Application Publication
(10) Pub. No.: US 2022/0162568 A1
(43) Pub. Date: May 26, 2022

(54) PORE

(71) Applicants: Oxford Nanopore Technologies Limited, Oxford (GB); P&Z Biological Technology, Newark, NJ (US)

(72) Inventors: Farzin Haque, Newark, NJ (US); Shaoying Wang, Newark, NJ (US); Lakmal Nishantha Jayasinghe, Oxford (GB); Michael Jordan, Oxford (GB)

(73) Assignees: Oxford Nanopore Technologies Limited, Oxford (GB); P&Z Biological Technology, Newark, NJ (US)

(21) Appl. No.: 17/602,339

(22) PCT Filed: Apr. 9, 2020
(86) PCT No.: PCT/GB2020/050923

§ 371 Date: Oct. 8, 2021

Related U.S. Application Data
(60) Provisional application No. 62/831,671, filed on Apr. 9, 2019.

(51) Int. Cl. C12N 7/00; C07K 14/005; G01N 33/68; G01N 33/487

(52) U.S. Cl. CPC C12N 7/00; C07K 14/005; B82Y 15/00; G01N 33/48721; C12N 2795/10222; G01N 33/6872

(57) ABSTRACT

A modified portal protein of a bacteriophage DNA packaging motor, wherein the modified portal protein is capable of direct insertion into a membrane and wherein the portal protein is modified compared to the wild type portal protein such that one or more amino acid residues on the outer surface of the portal protein is substituted by one or more other amino acid residue, and/or wherein a one or more amino acid residue is inserted on the outer surface of the portal protein so as to alter the outer surface hydrophobicity of the modified portal protein compared to the wild type portal protein.

### RELATED APPLICATIONS

This application is a national stage filing under 35 U.S.C. § 371 of international PCT application PCT/GB2020/050923, filed Apr. 9, 2020, which claims priority under 35 U.S.C. § 119(e) to U.S. provisional patent application, U.S. Ser. No. 62/831,671, filed Apr. 9, 2019, the entire contents of each of which are which is incorporated herein by reference.

### FIELD

The present disclosure relates to modified portal proteins, membranes comprising the modified portal proteins, and methods of characterising analytes using the membranes comprising the modified portal proteins.

### BACKGROUND

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Analytes can be organic and inorganic small molecules as well as various biological or synthetic macromolecules and polymers including polynucleotides, polypeptides and polysaccharides. Nanopore sensing can reveal the identity and perform single molecule counting of the sensed analytes, but can also provide information on the analyte composition such as nucleotide, amino acid or glycan sequence, as well as the presence of base, amino acid or glycan modifications such as methylation and acylation, phosphorylation, hydroxylation, oxidation, reduction, glycosylation, decarboxylation, deamination and more. Nanopore sensing has the potential to allow rapid and cheap polynucleotide sequencing, providing single molecule sequence reads of polynucleotides of tens to tens of thousands bases length.

Nanopores of biological origin are based on naturally occurring membrane proteins and can be inserted into a copolymer membrane by contacting the membrane with the purified protein and applying a voltage potential to the membrane.

The phi29 bacteriophage gp10 portal protein assembles into a propeller-like structure from 12 subunits of gp10. It has an external diameter of 14.6 nm and a height of 7.5 nm. At the narrowest constriction, the wild-type channel is 3.6 nm. Each of the 12 subunits has an elongated shape harboring a central α-helical domain composed of a three-helix bundle, an α-β motif, and a 6-fold stranded SH3-like domain at the wider C-terminus. The portal protein is not a natural membrane protein or ion channel, but has been proposed as a nanopore for characterising analytes. In order to be inserted into membranes, the bacteriophage phi29 portal protein must first be inserted into liposomes, which are then fused with planar lipid bilayers.

### SUMMARY

Disclosed herein are modified bacteriophage portal proteins that spontaneously insert into membranes. The inserted portal proteins can serve as nanopores.

In one aspect, a modified portal protein of a bacteriophage DNA packaging motor is provided that is capable of direct insertion into a membrane, wherein one or more amino acid residue on the outer surface of the portal protein is substituted by one or more other amino acid residues, and/or one or more amino acid residue is inserted on the outer surface of the portal protein, to alter the outer surface hydrophobicity of the portal protein compared to the wild type portal protein. The introduction, by substitution and/or insertion, of one or more amino acid residues may increase or decrease the outer surface hydrophobicity compared to the wild type portal protein. The outer surface hydrophobicity of a particular region of the protein may be increased or decreased.

Since the portal proteins channel assembles from 12 subunits, altering one or more residues in one monomer will trigger the effect in the entire channel with the mutation present in the same plane of the molecule. Overall, the portal protein is composed of two domains: wing and stalk domains. The stalk domain comprises a hydrophobic belt region underneath the wing of the portal protein.

In one embodiment, at least one of the one or more introduced amino acid residues is in the central hydrophobic belt region of the portal protein. The one or more introduced amino acid residues may be introduced by e.g., substitution and/or insertion. Residues in the hydrophobic belt region of the portal protein of the Phi29 DNA packaging motor include F24, 125, L28, F60, F128, P129 and P132. In one embodiment, an amino acid within one or two residues of any one or more of these positions, or at one or more corresponding positions in an analogous portal protein, may be substituted with one or more amino acid that is more hydrophobic than the amino acid naturally present at the substituted position. In one embodiment, a hydrophobic amino acid may be inserted within one or two residues of any one or more of these positions, or at one or more corresponding positions in an analogous portal protein. The N-terminal residues of each subunit of the portal protein are in the hydrophobic belt region. In one embodiment, at least one of the one or more amino acid residues is within 30 amino acids of the N-terminus of the portal protein. For example, at least one of the one or more amino acid residues is at a position corresponding to R10, E14, R17, Q18 and/or R22 of the portal protein of the Phi29 DNA packaging motor, or at a corresponding position within an analogous portal protein.

In one embodiment at least one of the one or more introduced amino acid residues is in the hydrophilic cis- and/or trans-layer of the portal protein. Examples of amino acid residues in the cis-layer of the portal protein include at a position corresponding to Q32, Y36, F52, K55, Q59, F60, Y62, N77, G78, A79, L80, S81, R84, R94, A96, S97, P98 and Q101in the wing domain of the portal protein of the Phi29 DNA packaging motor. Examples of amino acid residues in the trans-layer of the portal protein is at a position corresponding to P129, T131, E135, Q168 in the stalk domain of the portal protein of the Phi29 DNA packaging motor.

In a particular embodiment, at least one of the one or more introduced amino acid residues in the cis- or trans-layer of the portal protein at a position corresponding to A79, E135 and/or Q168 of the portal protein of the Phi29 DNA packaging motor is modified.

In one aspect, a modified portal protein of a bacteriophage DNA packaging motor is provided that is capable of direct insertion into a membrane, wherein one or more amino acid residues is introduced on the outer surface of the portal protein, to introduce a binding site on the outer side of the wing domain or in the stalk domain for a molecule that alters the hydrophobicity of the outer surface of the portal protein compared to the wild type portal protein. The binding site may be introduced by substitution of a residue present on the surface of the portal protein with another amino acid residue, or by insertion one or more amino acid residue.

In one embodiment, the at least one of the one or more amino acid residues introduced into the portal protein to introduce a binding site on the outer side of the wing domain or in the stalk domain is cysteine and/or a non-natural amino acid.

In one embodiment at least one of the one or more amino acid residues introduced into the portal protein to introduce a binding site is in the hydrophilic cis- and/or trans-layer of the portal protein. Examples of amino acid residues in the cis-layer of the portal protein include those at positions corresponding to one or more of Q32, Y36, F52, K55, Q59, F60, Y62, N77, G78, A79, L80, S81, R84, R94, A96, S97, P98 or Q101 in the wing domain of the portal protein of the Phi29 DNA packaging motor. Examples of amino acid residues in the trans-layer of the portal protein include those at a position corresponding to P129, T131, E135 or Q168 in the stalk domain of the portal protein of the Phi29 DNA packaging motor.

In a particular embodiment, at least one of the one or more amino acid residues in the cis- or trans-layer of the portal protein at a position corresponding to A79, E135 and/or Q168 of the portal protein of the Phi29 DNA packaging motor is modified to introduce a binding site.

In one embodiment, the molecule that alters the hydrophobicity of the outer surface of the portal protein compared to the wild type portal protein is a hydrophobic molecule. Exemplary hydrophobic molecules are those comprising porphrin, tetraphenylporphyrin, protoporphyrin IX, octaethylporphyrin, cholesterol, heme or biliverdin.

In some embodiments, the modified portal protein is modified by the addition and/or deletion of one or more amino acid residues at the N-terminus of the portal protein.

In certain embodiments, the modified portal protein is a modified portal protein of a DNA packaging motor from a bacteriophage selected from the group consisting of phi29, T3, T4, T5, T7, SPP1, HK97, Lamda, G20c, P2, P3 and P22.

In one embodiment, the modified portal protein is composed of identical subunits.

In other aspects the following are provided:
a subunit of a modified portal protein as disclosed herein;
a membrane comprising a modified portal protein as disclosed herein;
an array comprising two or more membranes comprising a modified portal protein as disclosed herein;
a device comprising an array comprising two or more membranes each comprising a modified portal protein as disclosed herein, a means for applying a potential across the membranes and a means for detecting electrical charges across the membranes; and
a method of characterising a target analyte, the method comprising contacting a membrane comprising a modified portal protein as disclosed herein with the target analyte and applying a potential across the membrane such that the target analyte moves with respect to the nanopore, and taking one or more measurements as the target analyte moves with respect to the pore, thereby determining the presence, absence or one or more characteristics of the analyte.

In one embodiment, the membrane is a lipid membrane or a copolymer membrane, such as a diblock or triblock copolymeric membrane.

In one embodiment, the array is adapted for insertion into a sensor device.

In one embodiment, the device further comprises a fluidics system configured to supply a sample to the membranes.

In one embodiment, the method comprises taking electrical measurements and/or optical measurements. In one embodiment of the method, multiple target analytes are characterised. In one embodiment of the method, the target analyte is a polynucleotide, protein, peptide, carbohydrate, metabolite or other chemical. In one embodiment of the method, the target analyte is associated with a medical condition.

### DESCRIPTION OF THE FIGURES

FIG. 1 shows the structure of the phi29 gp10 portal protein from different angles. The structure of a subunit (monomer unit) of the pore is also shown. A representative location to generate conjugation sites on the phi29 gp10 portal protein surface for incorporation of hydrophobic group is shown in one monomer of the protein. A. Side view of the wild-type phi29 gp10 pore. The region of interest where protein engineering is necessary for direct membrane insertion is boxed. The residues are present in either wing or stalk domains, as shown in B. Two distinct domains of the pore are shown the monomer unit. B. A monomer unit (one of 12 assembled subunits) of the assembled pore is shown. The wing domain is shown in black (residues 1-125) and the stalk domain in grey (residues 120-309). The region of interest which is boxed in panel A encompasses parts of both protein domains. C. Representative locations on WT phi29 gp10 protein to generate conjugation sites for incorporation of hydrophobic membrane anchoring modules. Specific points of mutation in the wing domain include residues Q32, Y36, F52, K55, Q59, F60, Y62, N77, G78, A79, L80, S81, R84, R94, A96, S97, P98 and Q101; Specific points of mutation in the stalk domain include P129, T131, E135 and Q168. D. Chart showing hydrophobicity scale of 20 natural amino acids. Typically, I, V L or F are introduced at target points of mutation to enhance the relative hydrophobicity of the regions of interest.

FIG. 2 shows representative locations to generate a cysteine mutation on the phi29 gp10 portal protein surface for incorporation of hydrophobic group. These locations include A79C in the wing domain, and E135C and Q168C in the stalk domain. Other locations not shown in this figure include Q32, Y36, F52, K55, Q59, F60, Y62, N77, G78, L80, S81, R84, R94, A96, S97, P98, Q101 in the wing domain; and P129, T131 in the stalk domain. Since the protein assembles as a dodecamer, each mutation generates a ring. The cysteine residue enables linkage of hydrophobic modules via standard sulfhydryl chemistry. Instead of cysteine, any unnatural amino acid can also be incorporated, such as amino acids with alkyne side chains for ‘click’-chemistry mediated chemical conjugation.

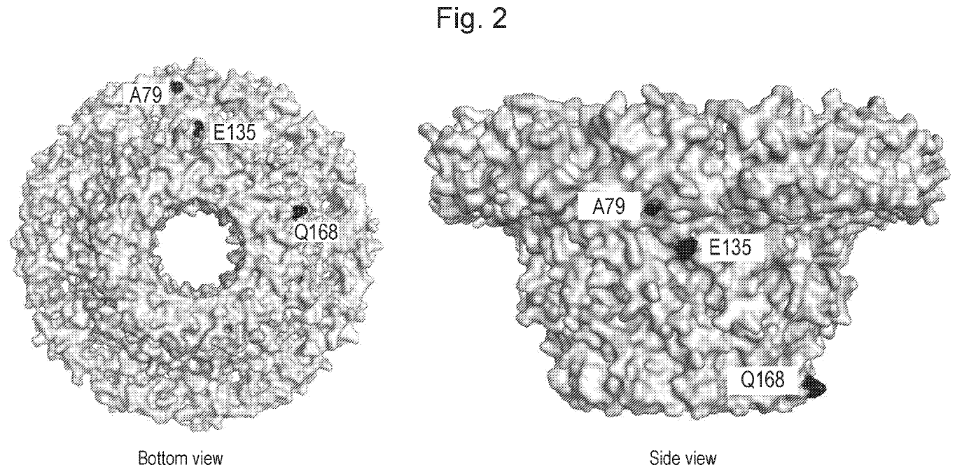

FIG. 3 shows a representative location to generate hydrophobic mutations on the phi29 gp10 portal protein surface to facilitate the insertion into polymer membrane. These locations include R10, E14, R17, Q18 and R22 in the wing domain close to the N-terminus of the subunit. These resides are typically mutated with hydrophobic residues I, V, L or F. Since the protein assembles as a dodecamer, each mutation generates a ring of hydrophobic residues along the plane. The location of the hydrophobic amino acid or hydrophobic anchoring module relative to the membrane core determines the position where the pore sits in the membrane.

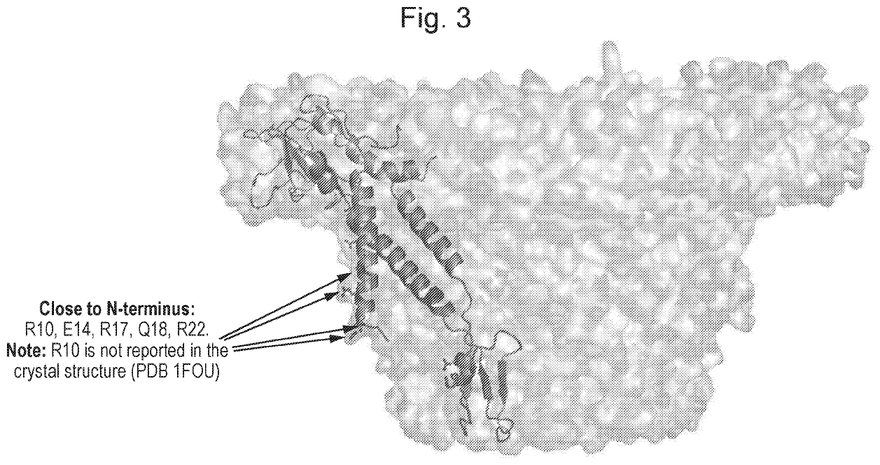

FIG. 4 is an SDS-PAGE gel showing that representative phi29 gp10 portal protein mutant A79C was expressed and purified. The mutant A79C gene was cloned into an expression vector and then transformed into E. coli. The successfully transformed bacteria were cultured in LB medium overnight. Protein expression was induced by adding IPTG. The bacteria were collected after induction and then lysed. The protein and other components were differentiated by centrifugation. An Ni-NTA His bind resin with a His tag was applied to purify the mutant protein. The protein was eluted using elution buffer containing increasing concentrations of imidazole, as shown in the figure. The eluent was collected and concentrated followed by FPLC purification. An SDS-PAGE gel was run to check the protein samples.

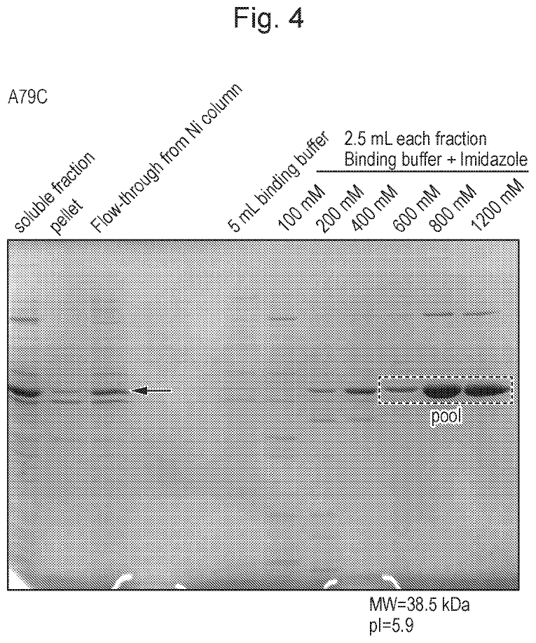

FIG. 5 is an SDS-PAGE gel showing that representative phi29 gp10 portal protein mutant E135C was expressed and purified. The mutant E135C gene was cloned into an expression vector and then transformed into E. coli. The successfully transformed bacteria were cultured in LB medium overnight. Protein expression was induced by adding IPTG. The bacteria were collected after induction and then lysed. The protein and other components were differentiated by centrifugation. An Ni-NTA His bind resin with a His tag was applied to purify the mutant protein. The protein was eluted using elution buffer containing increasing concentrations of imidazole, as shown in the figure. The eluent was collected and concentrated followed by FPLC purification. An SDS-PAGE gel was run to check the protein samples.

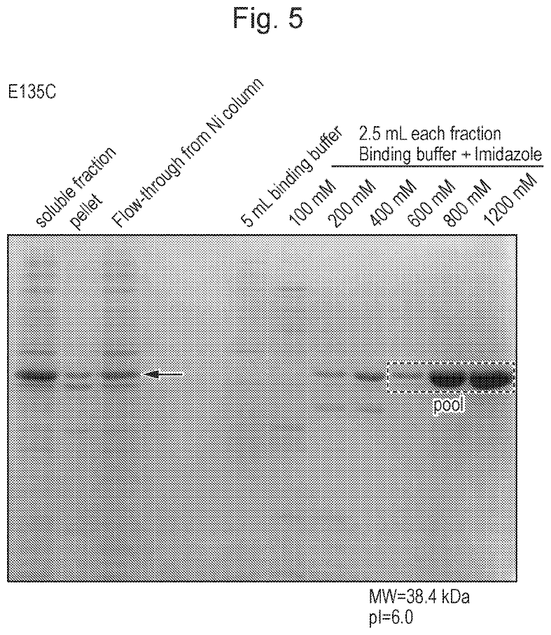

FIG. 6 is an SDS-PAGE gel showing that of representative phi29 gp10 portal protein mutant Q168C was expressed and purified. The mutant Q168C gene was cloned into an expression vector and then transformed into E. coli. The successfully transformed bacteria were cultured in LB medium overnight. Protein expression was induced by adding IPTG. The bacteria were collected after induction and then lysed. The protein and other components were differentiated by centrifugation. An Ni-NTA His bind resin with a His tag was applied to purify the mutant protein. The protein was eluted using elution buffer containing increasing concentrations of imidazole, as shown in the figure. The eluent was collected and concentrated followed by FPLC purification. An SDS-PAGE gel was run to check the protein samples.

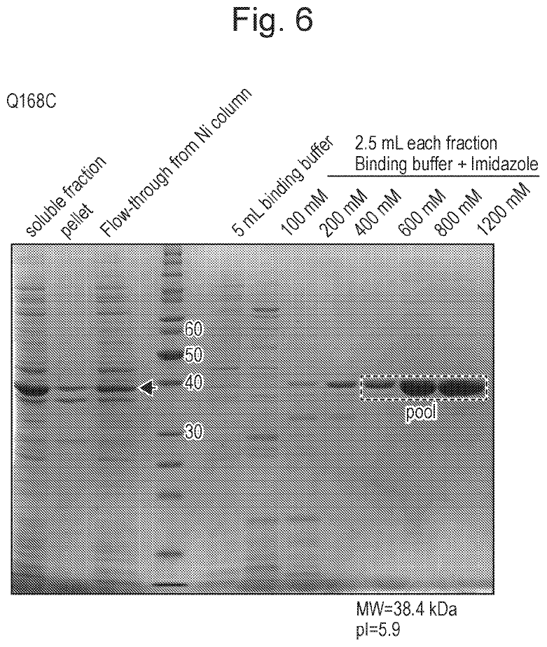

FIG. 7 is an SDS-PAGE gel showing that representative phi29 gp10 portal protein mutants R10L, E14V, R17L and N-7Δ (mutant-b) (left of the marker in the figure) and R10L, E14V, R17L, Q18L, R22I and N-7Δ (mutant-c) (right of the marker in the figure) were expressed and purified. The mutant genes were cloned into an expression vector and then transformed into E. coli. The successfully transformed bacteria were cultured in LB medium overnight. Protein expression was induced by adding IPTG. The bacteria were collected after induction and then lysed. The protein and other components were differentiated by centrifugation. An Ni-NTA His bind resin with a His tag was applied to purify the mutant protein. The protein was eluted using elution buffer containing increasing concentrations of imidazole, as shown in the figure. The eluent was collected and concentrated followed by FPLC purification. An SDS-PAGE gel was run to check the protein samples.

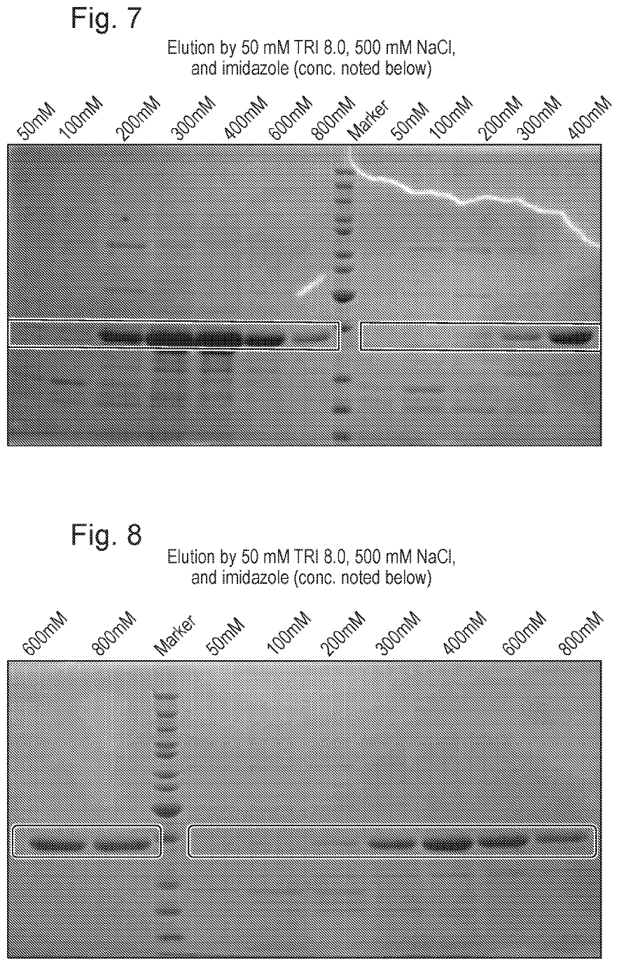

FIG. 8 is an SDS-PAGE gel showing that of representative phi29 gp10 portal protein mutants N-I-L (mutant-d) (left of the marker in the figure) and R10L, E14V, R17L, N-ter-7Δ with I-L added to the N-ter (mutant-e) (right of the marker in the figure) were expressed and purified. The mutant genes were cloned into an expression vector and then transformed into E. coli. The successfully transformed bacteria were cultured in LB medium overnight. Protein expression was induced by adding IPTG. The bacteria were collected after induction and then lysed. The protein and other components were differentiated by centrifugation. An Ni-NTA His bind resin with a His tag was applied to purify the mutant protein. The protein was eluted using elution buffer containing increasing concentrations of imidazole, as shown in the figure. The eluent was collected and concentrated followed by FPLC purification. An SDS-PAGE gel was run to check the protein samples.

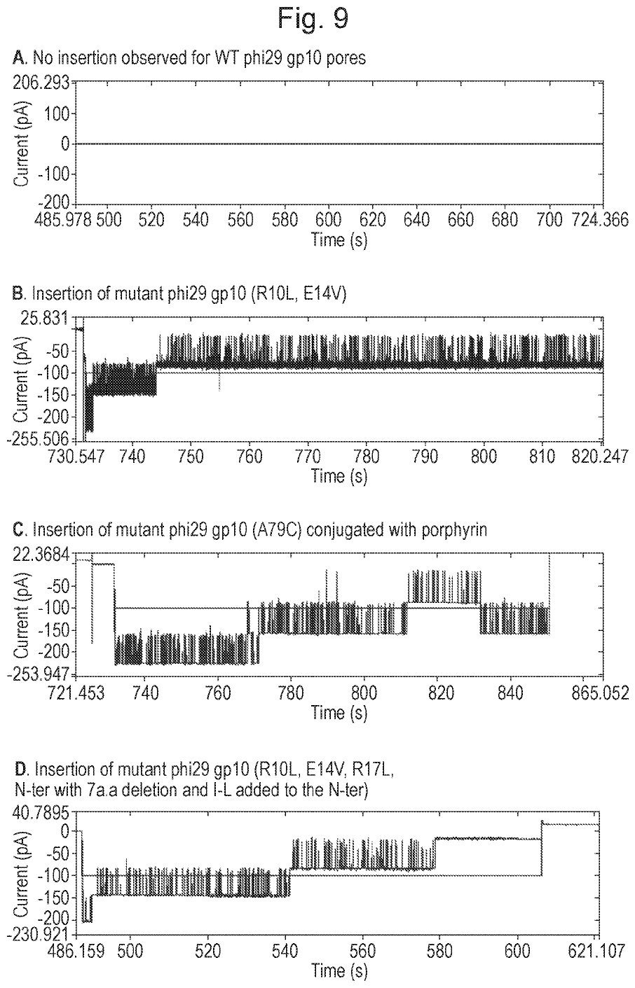

FIG. 9 shows data obtained using the engineered mutants of phi29 gp10 portal protein in an Oxford Nanopore Technologies MinION device. A. No direct insertion in ONT membranes observed with WT phi29 gp10 pores. B-F. Direct insertion of engineered phi29 gp10 pores in ONT membranes: B. mutant (R10L, E14V); C. mutant A79C with conjugated porphyrin; D. mutant (R10L, E14V, R17L, N-terminus with 7 a.a. deleted and I-L tag added); E. mutant (R10L, E14V, R17L, N-terminus with 7 a.a. deleted); F. mutant (Q168C) with conjugated cholesterol. To insert the engineered protein channel into ONT membranes, protein with 1 mg/ml concentration was diluted 1000-fold in C13 buffer (25 mM potassium phosphate, 150 mM potassium ferrocyanide, 150 mM potassium ferricyanide, pH 8). 200 ul diluted protein sample was added through the priming port of the MinION flowcell. Then a ramping voltage from +50 to +350 mV (5 mV increments; 20 s holding) was applied to assist the insertion of the protein channel. The flow cell was then flushed with 2 mL C13 buffer. An I-V curve was then run typically, ±50, ±100, ±150, ±200 mV with variable holding times (2 mins to 10 minutes holding at each voltage) to observe pore behavior over time. Analytes such as DNA or peptide (1 pM concentration) was suspended in C13 buffer and added to the flow cell to check pore functionality. In A-F, Voltage applied—100 mV. Conduction buffer: C13 (ONT reagent). Analyte—TAT peptide which gives rise to distinctive current blockage events—indicative of a functional pore. G. Relative insertion rate of WT and engineered mutants. The rate is relative to the WT. There are variations for different mutants within each noted category, but the overall trend is as shown in the chart.

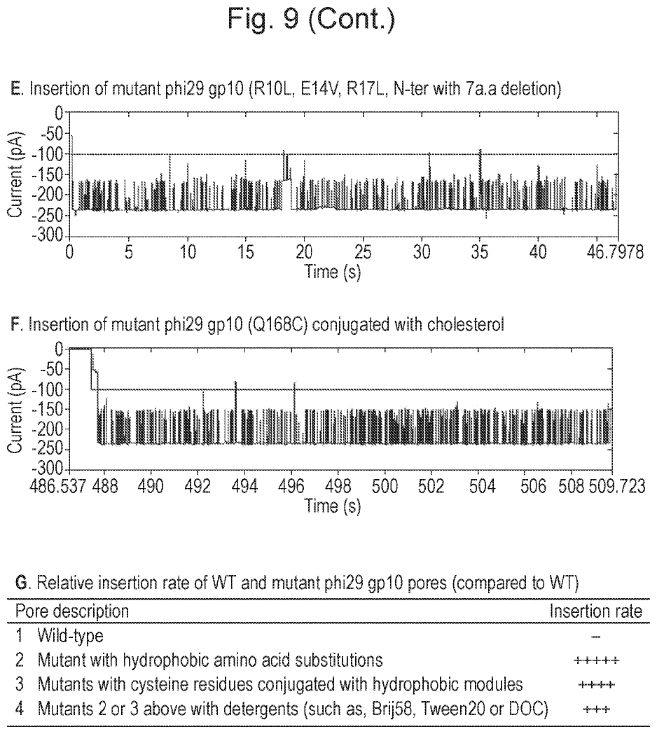

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e. Q42R means that Q at position 42 is replaced with R.

### Portal Proteins

The nanopore is a modified portal protein of a viral DNA packaging motor, such as a bacteriophage DNA packaging motor.

The portal protein of a bacteriophage DNA packaging motor has a truncated cone structure. The protein has a central channel formed by twelve portal protein subunits, also referred to as connector protein subunits. An exemplary unmodified viral DNA-packaging motor portal protein from bacteriophage phi29 has been purified and its three-dimensional structure has been crystallographically characterized (e.g., Guasch et al., 1998 FEBS Lett. 430:283; Marais et al., 2008 Structure 16:1267). The phi29 channel has a 3.6 nm narrow and a 6 nm wide end, which is larger than most membrane protein channels. Accordingly, a number of embodiments as described herein refer to the phi29 DNA-packaging gp10 motor portal protein (e.g., Genbank Accession No. ACE96033 UniProt ID: P04332; Gene ID: 6446518; SEQ ID NO: 1) and/or to polypeptide subunits thereof including fragments, variants and derivatives thereof that are capable of forming a channel (e.g., Accession Nos. gi 29565762, gi 31072023, gi 66395194, gi 29565739, gi 157738604).

While the portal proteins of viruses share little sequence homology and vary in molecular weight, there is significant underlying structural similarity. In particular, DNA-packaging motor connector proteins of other dsDNA viruses (e.g., T4, lambda, P22, P2, T3, T5 and T7), despite sharing little sequence homology with, and differing in molecular weight from, the phi29 connector, exhibit significant underlying structural similarities (e.g., Bazinet et al., 1985 Ann Rev. Microbiol. 39:109-29).

In certain embodiments, the use of an isolated viral DNA-packaging motor portal protein from other dsDNA viruses is contemplated, including without limitation the isolated viral DNA-packaging motor portal protein from any of phage lambda, P2, P3, P22, T3, T4, T5, SPP1, HK97 and T7, such as an isolated dsDNA virus DNA-packaging motor portal protein (e.g., T4 (Accession No. NP-049782)(Driedonks et al., 1981 J Mol Biol 152:641), lambda (Accession Nos. gi 549295, gi 6723246, gi 15837315, gi 16764273)(Kochan et al., 1984 J Mol Biol 174:433), SPP1 (Accession No. P54309), P22 (Accession No. AAA72961)(Cingolani et al., 2002 J Struct Biol 139:46), G20c (Accession No. KX987127.1), P2 (Accession No. NP-046757, P3 (Nutter et al., 1972 J. Viral. 10(3):560-2), T3 (Accession No. CAA35152)(Carazo et al., 1986 Jl. Ultrastruct Mol Struct Res 94:105), T5 (Accession numbers AAX12078, YP-006980; AAS77191; AAU05287), T7 (Acc. No. NP-041995)(Cerritelli et al., 1996 J Mol Biol 285:299; Agirrezabala et al., 2005 J Mol Biol 347:895)). In some embodiments, the connector protein comprises bacteriophage T3 connector protein gp8. In some embodiments, the connector protein comprises bacteriophage T7 connector protein gp8. In some embodiments, the connector protein comprises bacteriophage T4 connector protein gp20. In some embodiments, the connector protein comprises bacteriophage T5 connector protein gp7. In some embodiments, the connector protein comprises bacteriophage SPP1 connector protein gp6. In some embodiments, the connector protein comprises bacteriophage HK97 connector protein gp3.

Like the phi29 DNA-packaging motor portal protein exemplified herein, these and other dsDNA virus packaging motor portal proteins, which have been substantially structurally characterized, can be modified such that they are incorporated into a membrane layer to form an aperture through which conductance can occur when an electrical potential is applied across the membrane in the same manner as the portal protein of the phi29 DNA-packaging motor. Accordingly, disclosure herein with respect to the phi29 portal protein is intended to be illustrative of related embodiments that are contemplated using any of such other isolated dsDNA viral DNA-packaging motor portal proteins.

The portal protein of the phi29 DNA-packaging motor, or the portal protein from another bacteriophage DNA-packaging motor, may be modified according to the teachings found herein.

Isolated DNA-packaging motor portal proteins that have been artificially engineered to possess properties of membrane incorporation (e.g., stable transmembrane integration in a membrane layer) according to the present disclosure can be used as electroconductive biosensors for cancer biomarkers. The portal proteins may also be artificially engineered to influence the electroconductive properties of the transmembrane channel formed by the portal proteins.

Modified isolated double-stranded DNA virus DNA-packaging motor protein connectors such as the phi29 connector may be engineered to have desired structures for use in the presently disclosed embodiments, where protein crystallographic structural data are readily available. Procedures for large scale production and purification of phi29 connector have been developed (Guo et al., 2005; Ibanez et al., Nucleic Acids Res. 12, 2351-2365 (1984), Robinson et al., Nucleic Acids Res. 34, 2698-2709 (2006), Xiao et al., ACS Nano 3, 100-107 (2009).

In one embodiment, a modified bacteriophage phi29 viral DNA-packaging motor portal protein (e.g. SEQ ID NO: 1) has at least 80%, 90%, or 95% identity to the wild type protein, or to a portion of a wild-type phi29 viral DNA-packaging motor connector protein-derived, which portion contains at least 150, 175, 200, 225, 250, 275, including at least 240, 260, 280, 285, 290, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330 or more amino acids.

In other embodiments, the modified portal protein is a modified double-stranded DNA portal proteins from another bacteriophage, such as phage T4, lambda phage (Accession numbers gi549295, gi6723246, gi15837315, gi16764273), phage SPP1 (Accession number P54309), phage P22 (Accession number AAA72961), phage P2 (Accession number NP-046757), phage P3 (Nutter et al., 1972 J. Virol. 10(3):560-2), phage T3 (Accession number CAA35152), phage T5 (Accession numbers AAX12078, YP006980; AAS77191; AAU05287), phage T7 (Accession number NP041995) and phage HK97 (Accesssion number NP_037699). For example, the modified portal protein may be a mutant of any of these bacteriophage viral DNA-packaging motor portal proteins, and may, for example, have at least 80%, 90%, or 95% identity to the herein disclosed polypeptides and to fragments of such polypeptides. A “fragment” of a mutant portal protein subunit generally contains at least 150, 175, 200, 225, 250, 275, including at least 240, 260, 280, 285, 290, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330 or more amino acids.

The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The portal protein may be a modified analogue of any of the above bacteriophage portal proteins. The “analogue” when referring to viral DNA-packaging motor portal proteins means a naturally occurring homologue or a variant of a viral DNA-packaging motor portal protein. The portal protein is typically composed of subunits that are capable of self-assembly into oligomeric, for example a homododecameric, channel.

The portal protein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which additional amino acids are genetically fused to one or more portal protein subunit, including amino acids that are employed for detection or specific functional alteration of the mutant portal protein.

The modified portal protein is isolated. The term “isolated” means that the material is removed from its original environment (e.g., the natural environment if it is naturally occurring). For example, a naturally occurring protein present in a an intact naturally occurring virus is not isolated, but the same protein, separated from some or all of the co-existing materials in the natural system, is isolated. Such proteins could be part of a composition, and still be isolated in that such vector or composition is not part of its natural environment. Methods of isolating portal proteins of bacteriophage motor proteins are known in the art. The portal proteins of bacteriophage motor proteins for use in the methods and compositions described herein can be produced recombinantly used methods well known in the art.

In one embodiment, the modified protein is a truncated version of the portal protein, and/or the modified protein may comprise additional amino acids at one or both ends of one or more of the subunits of the portal protein, and/or may comprise one or more amino acid substitution, deletion or addition within the amino acid sequence of the portal protein.

The truncated portal protein may be truncated at the N-terminus and/or the C-terminus. For example, up to about 30 amino acids may be deleted from the N-terminus, such as up to about 20, 10, 9, 8 or 7 amino acids may be deleted from the N-terminus. Alternatively or additionally, up to about 30 amino acids may be deleted from the C-terminus, such as up to about 20, 10, 9, 8 or 7 amino acids may be deleted from the C-terminus. One or more, such as from 2 to about 30 amino acids, such as from 3 to about 20, 4 to about 10, 5 to 9 or 6, 7 or 8 amino acids, may be added to the N-terminus and/or to the C-terminus, or to the truncated N-terminus and/or the truncated C-terminus.

The modified portal protein comprises a channel. In one embodiment, the portal protein is modified to alter one or more property of the channel of the nanopore. In one embodiment, this is achieved by modifying one or more of the amino acid residues lining the channel, and/or at the entrance to the channel.

In some embodiments, the nanopore comprises only full length subunits of the portal protein.

In some embodiments, the nanopore is a multimeric protein formed of six or more portal protein subunits, such as 7, 8, 9 10, 11 or 12 subunits. For example, the nanopore may be a dodecameric protein. One or more, such as 2, 3, 4, 5, 6, 7, 8, 9, 10, 11 or 12 of the subunits may be modified. In one embodiment, one or more of the subunits may be modified at the C-terminus and/or N-terminus, such as, for example, to increase the hydrophilicity at one or both ends of the nanopore. For example, one or more of the subunits may be modified by the addition of a flexible linker and/or a peptide tag at the C-terminus and/or N-terminus. In some embodiments, the nanopore is composed of identical subunits. Any suitable linker may be used, such as, for example, a linker comprising from 3 to 12 amino acids, such as from 4 or 5 to 10, preferably 6 to 8 amino acids. The amino acids in the linker may selected from lysine, serine, arginine, proline, glycine, alanine aspartic acid, tyrosine, isoleucine and/or threonine. Examples of suitable linkers include, but are not limited to, the following: GGGS, PGGS, PGGG, RPPPPP, RPPPP, VGG, RPPG, PPPP, RPPG, PPPPPPPPP, RPPG, GGG, GGGG, GGGGG, GGGGGG and DYDIPTT.

The modified portal protein may comprise a tag, for example to facilitate its purification. Any suitable peptide tag may be used to facilitate purification of the portal protein. For example, in one embodiment, the tag may be a strep tag. In one embodiment, the streptag has a length of from 8 to 11 amino acids and/or the streptag amino acid sequence contains the motif HPQ. The streptag may for example comprise or consist of the amino acid sequence WSHPQSEK, WSHPQFEK, NWSHPQFEK, PWSHPQFEK or GGSHPQFEG. This sequence may be varied by addition, deletion or substitution of one or more, such as 2, 3, 4 or 5 of the amino acids, provided that the core “HPQ” motif is maintained. The variant sequence is typically from 8 to 11 amino acids NWSHPQFEK, PWSHPQFEK, and GGSHPQFEG. In another embodiment, the tag may be a His-tag (typically His6 (HHHHHH)).

The portal protein may include a cleavage site to allow the tag and/or linker to be removed from the subunit before or after assembly of the pore. Any suitable cleavage site may be used. One example is a TEV (Tobacco Etch Virus) clearage site (ENLYFQG; with cleavage occurring between the Q and G residues).

### Modification by Introducing Amino Acids to Facilitate Insertion

In one aspect, the portal protein is modified to facilitate its direct insertion into a membrane by introducing one or more amino acids to alter the hydrophobicity of the surface of the pore. The one or more amino acids may be introduced by substitution and/or insertion. The inserted amino acids may be inserted at one or both ends of the amino acid chain of a portal protein subunit, and/or between two amino acids in the chain. When the subunit is folded and assembled into a pore, the introduced amino acid is present on the outer surface of the pore.

The introduction is made to at least one amino acid in one or more of the subunits in the pore. Each subunit in the pore may independently comprise one or more introduced amino acid, such as 2, 3, 4, 5, 6, 7, 8, 9, 10 or more amino acid introductions. The pore may be comprised of identical modified subunits but one or more of the subunits in the pore may be different from the others. The pore may, for example, be composed of two or more different subunits, such as from 3 or more, different subunits. For example, 4, 5, 6, 7, 8, 9, 10 or 11 different modified subunits may be present in the pore. In one embodiment, all of the subunits may be different from each other. Typically, at least one amino acid is introduced into each subunit in the pore. For example, where the pore comprises 12 subunits, the pore comprises twelve or more introduced or substituted amino acids to alter the hydrophobicity of the surface of the pore.

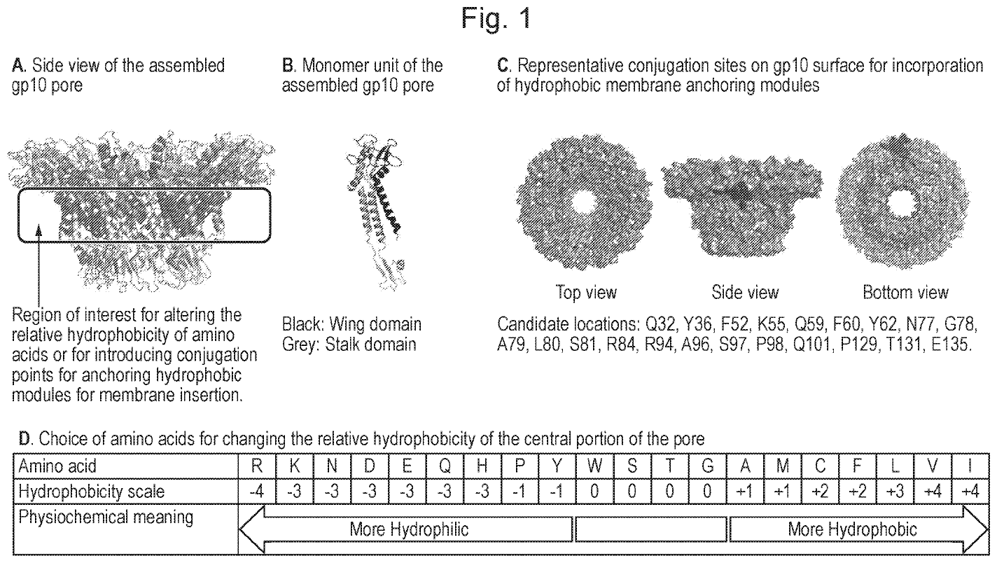

The modifications are typically made in the central hydrophobic belt around the outside of the pore. The location of the central hydrophobic belt region is shown in FIG. 1. The central belt region of the pore is typically modified to increase its hydrophobicity. An increased hydrophobicity may, for example, be achieved by substituting hydrophilic, neutral or relatively less hydrophobic amino acids (such as, for example, alanine and/or methionine) with more hydrophobic residues (such as, for example, leucine, valine and/or isoleucine). FIG. 1D shows the relative hydrophobicity of amino acids. The substitution to increase hydrophobicity may be substitution of one or more amino acids present in the pore with any amino acid having a more positive number on the hydrophobicity scale as shown in FIG. 1D than the amino acid being replaced.

An increase in hydrophobicity may be achieved by inserting one or more hydrophobic amino acids into and/or at the ends of the amino acid chain of a portal protein subunit. Hydrophobic amino acids are shown in FIG. 1D.

The location of the introduced hydrophobic amino acid can determine the position in which the pore sits in the membrane. The position of the pore relative to the membrane can be shifted up or down (for example by up to 0.5 nm in either direction). The position of the pore in the membrane can, therefore, be controlled to improve the stability of the pore in the membrane. The inherent electrophysiology of the pore is typically not changed by the alteration of the amino acids on the outside surface of the pore.

In one embodiment, hydrophobicity is altered by introducing one or more amino acids in the belt region underneath the wing domain. Target locations include Phe24, Ile25, Leu28, Phe60, Phe128, Pro129, and Pro132 in the phi29 portal protein subunit, and corresponding positions in analogous subunits. Additional hydrophobic residues may be substituted or inserted within one or two amino acids, either before and/or after, of any one or more of these target locations, such as 2, 3, 4, 5, 6, 7 or 8 of these target locations. For example, the hydrophobicity of the amino acid residues at positions corresponding to positions 22, 23, 26, 27, 29, 30, 58, 59, 61, 62, 126, 127, 130, 131, 133 and/or 134 in SEQ ID NO: 1 may be increased by substitution or insertion of amino acid resides. Changes may be made at any one or more, such as any 2, 3, 4, 5, 6, 7, 8, 9 or 10 or more of these positions. Other exemplary positions for mutation include positions corresponding to Arg10, Glu14, Arg17, Gln18, and Arg22 in SEQ ID NO: 1. Any one or more of these amino acids may be substituted with more hydrophobic residues to increase the hydrophobicity of the surface of the central region of the pore.

In one embodiment, exposed charge residues (such as residues corresponding to Arg17, Arg22 and/or Lys172 of SEQ ID NO: 1) in the stalk region as well as Asn/Gln residues (such residues corresponding to Asn166, Asn167, Gln168, Gln173, Asn176 and/or Gln177 in SEQ ID NO: 1) concentrated at the two distal portions of the protein stalk may be altered to change the hydrophilic properties.

Modification by Introducing Amino Acids to Facilitate Conjugation of Molecule that Alters Hydrophobicity

In one aspect, the modified portal protein of a bacteriophage DNA packaging motor that is modified so that it can be inserted directly into a membrane is one in which one or more amino acid residues on the outer surface of the portal protein are substituted by another amino acid residue and/or one or more amino acid residue is introduced on the outer surface of the portal protein, to introduce a binding site for a molecule that alters the hydrophobicity of the outer surface of the portal protein compared to the wild type portal protein. The binding site is introduced on the outer side of the wing domain or in the stalk domain. The binding site serves as a site of attachment for the molecule. The molecule is typically a hydrophobic molecule that increases the hydrophobicity of the surface of the portal proteins.

The introduced binding site may be, in one embodiment, a cysteine residue. In another embodiment, the binding site may be a non-natural amino acid.

A non-natural amino acid is an amino that is not naturally found in proteins. The non-natural amino acid is preferably not histidine, alanine, isoleucine, arginine, leucine, asparagine, lysine, aspartic acid, methionine, cysteine, phenylalanine, glutamic acid, threonine, glutamine, tryptophan, glycine, valine, proline, serine or tyrosine. The non-natural amino acid is more preferably not any of the twenty amino acids in the previous sentence or selenocysteine

Preferred non-natural amino acids for use in the invention include, but are not limited, to 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O-2-Propyn-1-yl-L-tyrosine, 4-(Dihydroxyboryl)-L-phenylalanine, 4-[(Ethylsulfanyl)carbonyl]-L-phenylalanine, (2S)-2-amino-3-{4-[(propan-2-ylsulfanyl)carbonyl]phenyl}propanoic acid, (2S)-2-amino-3-{4-[(2-amino-3-sulfanylpropanoyl)amino]phenyl}propanoic acid, O-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, 0-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2, T-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, O-(4,5-Dimethoxy-2-nitrobenzyl)-L-serine, (2S)-2-amino-6-({[R2-nitrobenzyl)oxy]carbonyl}amino)hexanoic acid, 0-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-1-naphthyl]sulfonylamino]propanoic acid, (2S)-2-amino-4-(7-hydroxy-2-oxo-2H-chromen-4-yl)butanoic acid, (2S)-3-[(6-acetylnaphthalen-2-yl)amino]-2-aminopropanoic acid, 4-(Carboxymethyl)phenylalanine, 3-Nitro-L-tyrosine, 0-Sulfo-L-tyrosine, (2R)-6-Acetamido-2-ammoniohexanoate, 1-Methylhistidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N⁶-{[(2R,3R)-3-Methyl-3,4-dihydro-2H-pyrrol-2-yl]carbonyl}-L-lysine, N⁶-[(Benzyloxy)carbonyl]lysine, (2S)-2-amino-6-[(cyclopentylcarbonyl)amino]hexanoic acid, N⁶[(Cyclopentyloxy)carbonyl]-L-lysine, (2S)-2-amino-6-{[(2R)-tetrahydrofuran-2-ylcarbonyl]amino}hexanoic acid, (2S)-2-amino-8-[(2R,3S)-3-ethynyltetrahydrofuran-2-yl]-8-oxooctanoic acid, N⁶-(tert-Butoxycarbonyl)-L-lysine, (2S)-2-Hydroxy-6-({[(2-methyl-2-propanyl)oxy]carbonyl}amino)hexanoic acid, N⁶-[(Allyloxy)carbonyl]lysine, (2S)-2-amino-6-({[(2-azidobenzyl)oxy]carbonyl}amino)hexanoic acid, N⁶-L-Prolyl-L-lysine, (2S)-2-amino-6-{[(prop-2-yn-1-yloxy)carbonyl]amino}hexanoic acid and N⁶[(2-Azidoethoxy)carbonyl]-L-lysine. The most preferred non-natural amino acid is 4-azido-L-phenylalanine (Faz).

Examples of suitable hydrophobic molecules that can be conjugated to the portal protein include porphyrin, tetraphenylporphyrin, protoporphyrin IX, octaethylporphyrin, cholesterol, heme and biliverdin. These and other hydrophobic molecules may be attached to the portal protein to enable membrane anchorage of the portal protein.

The exact location of the binding site for the hydrophobic molecule can be controlled to determine the position in which the pore sits in the membrane. The hydrophobic molecule can be used to shift the position of the pore relative to the membrane. For example, the pore can be shifted up or down in the membrane (for example by up to 0.5 nm in either direction) by the hydrophobic molecule. The stability of the pore in the membrane can thereby be controlled. The positioning of a hydrophobic molecule on the outside surface of the pore does not change the inherent electrophysiological properties of the pore.

Examples of locations where binding (or conjugation) sites can be introduced in the Phi29 Gp10 portal protein include Q32, Y36, F52, K55, Q59, F60, Y62, N77, G78, A79, L80, S81, R84, R94, A96, S97, P98, Q101, P129, T131, E135, Q168. Any one or more of the residues at these positions in the Phi29 Gp10 portal protein or corresponding positions in other portal proteins may be substituted by, for example, cysteine or a non-natural amino acid to introduce a binding side for a hydrophobic molecule. A cysteine residue or non-natural amino acid residue may alternatively be inserted within one or two residues of these positions.

In one embodiment, hydrophobicity is adjusted to facilitate insertion of the portal protein into a membrane by adding one or more natural or non-natural amino acids at one or both of the terminal ends of the subunit molecule. For example, a hydrophilic or hydrophobic tag may be added to one or both of the terminal ends. Typically, a hydrophobic tag is added to the N-terminal end which is present in the central belt region of the molecule and/or a hydrophilic tag may be added to the C-terminal domain. The hydrophilic and/or hydrophobic tag may be joined to the portal protein via a linker. Suitable linkers are described above.

The tag may comprise, for example, from two to twelve amino acids, such as from 3 or 4 to 10, for example 5, 6, 7, 8 or 9 amino acids. In one embodiment, all of the amino acids in the tag are hydrophilic amino acids. A hydrophilic amino acid is a n amino acid having a negative number on the hydrophobicity scale (as shown in FIG. 1D). One or more of the residues in the hydrophilic tag may be a residue at position 0 on the hydrophobicity scale. In one embodiment the hydrophilic tag may be hydrophilic overall, yet comprise one or more hydrophobic residues having a positive number on the hydrophobicity scale.

In an embodiment that uses a hydrophobic tag, the hydrophobic tag may, for example, include only residues having a positive number in the hydrophobicity scale. Alternatively, the hydrophobic tag may include one or more residues having a hydrophobicity of 0. Provided that the tag is hydrophobic overall, the tag may include one or more polar or charged amino acids having a negative number on the hydrophobicity scale.

### Mutations to Facilitate Use as Nanopore Sensor

The modified portal protein may include one or more additional modifications to alter other properties of the pore. Such alterations typically facilitate the use of the pore as a nanopore sensor. Examples of such modifications include the following:

Changing the overall electronegative property of the channel interiors by altering the rings of negatively charged Arg/Lys or Asp/Glu residues. Arg/Gly residuals may, for example be substituted by positively charged or neutral amino acids. One or more Asp/Glu residues may be substituted by positively charged, negatively charged or neutral amino acids. Altering the acidic residues at the inner channel entrance at the narrower end, such as Glu189, Asp19, and Asp194 in SEQ ID NO: 1, with any amino acids to change the hydrophilicity.

Adding several amino acids (any natural or non-natural) at the terminal ends with the goal of using these amino acids as anchoring point for added functionalities or for altering the electrophysiological properties of the pore.

Altering (deleting, truncating, mutating) the internal flexible loop, for example residues 229-244 in the phi29 Gp10 portal protein, to change the electrophysiological properties and/or detection capabilities of the pore.

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

The membrane is most preferably one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

Methods for Inserting Modified Pores into Membranes

Disclosed herein are methods for inserting modified portal proteins of bacteriophage DNA packaging motors into membranes for use as nanopores.

The modified portal proteins can be inserted into a copolymer membrane by contacting the membrane with the purified protein and applying a voltage potential to the membrane. Such methods are used in the art for inserting nanopores into membranes.

One exemplary method involves contacting the membrane with the modified portal protein and applying a ramping voltage to assist the insertion of the portal protein into the membrane to form a channel. The skilled person would readily be able to determine suitable portal protein concentrations and voltages. For example a ramping voltage of from +50 to +350 mV may be applied, with the voltage being increased by 5 mV increments, with, for example, a hold of about 20 seconds at each voltage. Prior to use as a sensor, excess portal protein can be washed away.

### Arrays

The disclosure provides an array of membranes comprising nanopores, wherein the nanopores are comprised of modified portal proteins. In a preferred embodiment, each membrane in the array comprises one nanopore. Due to the manner in which the array is formed, for example, the array may comprise one or more membrane that does not comprise a nanopore, and/or one or more membrane that comprises two or more nanopores. The array may comprise from about 2 to about 1000, such as from about 10 to about 800, from about 20 to about 600 or from about 30 to about 500 membranes.

In one embodiment, the array of membranes containing the modified portal protein nanopore may be present in a device suitable for high throughput sequencing.

### Sensor Device

The disclosure provides a device comprising an array of membranes containing the modified portal protein nanopore. For example, the device may comprise a chamber comprising an aqueous solution and a barrier that separates the chamber into two sections. The barrier typically has an aperture in which the membrane containing the nanopore is formed. Alternatively, the barrier may form the membrane in which the pore is present.

The device may thus comprise a first chamber and a second chamber, wherein the first and second chambers are separated by a membrane comprising a modified portal protein nanopore. When used to characterise a target polynucleotide, the device may further comprise a target polynucleotide, wherein the target polynucleotide is transiently located within the channel formed by the portal protein and wherein one end of the target polynucleotide is located in the first chamber and one end of the target polynucleotide is located in the second chamber.

In one embodiment, the device is capable of supporting the plurality of nanopores and membranes and operable to perform analyte characterisation using the nanopores and membranes. In one embodiment, the device comprises at least one port for delivery of the material for performing the characterisation. In one embodiment, the device comprises at least one reservoir for holding material for performing the characterisation. In one embodiment, the device comprises a fluidics system configured to controllably supply material from the at least one reservoir to the sensor device; and one or more containers for receiving respective samples, the fluidics system being configured to supply the samples selectively from one or more containers to the sensor device. The device may also comprise an electrical circuit capable of applying a potential and measuring an electrical signal across the membrane and pore complex.

The device may be any of those described in WO 2008/102120, WO 2009/077734, WO 2010/122293, WO 2011/067559 or WO 00/28312.

In one embodiment, the device forms part of a system for characterizing analytes. The system may, in one embodiment, comprise an electrically-conductive solution in contact with the nanopore, electrodes providing a voltage potential across the membrane, and a measurement system for measuring the current through the nanopore. In one embodiment, the voltage applied across the membrane and pore complex is from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different nucleotides by a pore by using an increased applied potential. Any suitable electrically-conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g. in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

A buffer may be present in the electrically-conductive solution. Typically, the buffer is phosphate buffer. Other suitable buffers are HEPES and Tris-HCl buffer. The pH of the electrically-conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The device may be compatible with a high throughput apparatus. For example, the device may be a SmidgION, MinION, GridION, PromethION instrument developed by Oxford Nanopore Technologies Ltd. These instruments can be fitted with different types of flow cells in which the nanopore is embedded in a copolymeric membrane. The device may be a flow cell. The copolymeric membrane is stable at least for a few months and also resistant to higher voltages. Each channel contains its own pair of electrodes, thus separating electrical signals between channels.

### Methods of Characterising an Analyte

In a further aspect, a method of determining the presence, absence or one or more characteristics of a target analyte is disclosed. The method involves contacting the target analyte with a membrane comprising a pore complex, such that the target analyte moves with respect to, such as into or through, the continuous channel comprising at least two constructions provided by a nanopore and an auxiliary protein or peptide in the pore complex, respectively, and taking one or more measurements as the analyte moves with respect to the channel and thereby determining the presence, absence or one or more characteristics of the analyte. The analyte may pass through the nanopore constriction, followed by the auxiliary protein constriction. In an alternative embodiment the analyte may pass through the auxiliary protein constriction, followed by the nanopore constriction, depending on the orientation of the pore complex in the membrane.

In one embodiment, the method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore complex, or in the vicinity of either opening of the channel will have an effect on the open-channel ion flow through the pore, which is the essence of “molecular sensing” of pore channels. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defense industry.

The target analyte may be a metal ion, an inorganic salt, a polymer, an amino acid, a peptide, a polypeptide, a protein, a nucleotide, an oligonucleotide, a polynucleotide, a polysaccharide, a dye, a bleach, a pharmaceutical, a diagnostic agent, a recreational drug, an explosive, a toxic compound, or an environmental pollutant. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

In one embodiment, the analyte is an amino acid, a peptide, a polypeptides or protein. The amino acid, peptide, polypeptide or protein can be naturally-occurring or non-naturally-occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

In a preferred embodiment, the analyte is a polynucleotide, such as a nucleic acid. A polynucleotide is defined as a macromolecule comprising two or more nucleotides. The naturally-occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are described in, for example, WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction (or “reading head”). It will be appreciated that sequencing may be performed upon an intact nucleic acid polymer that is ‘threaded’ through the pore via the action of an associated polymerase or helicase, for example. Alternatively, sequences may be determined by passage of nucleotide triphosphate bases that have been sequentially removed from a target nucleic acid in proximity to the pore (see for example WO 2014/187924).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer). The sequence of the nucleotides (iii) is determined by the consecutive identity of following nucleotides attached to each other throughout the polynucleotide strain, in the 5′ to 3′ direction of the strand.

The following Examples illustrate the invention.

### Example 1: Protein Expression and Purification

The engineered genes of phi29 portal protein channel was cloned into an expression vector. The newly constructed clones were transformed BL21 (DE3) E. coli bacteria. The successfully transformed bacteria were cultured in 10 mL Luria-Bertani (LB) medium overnight at 37° C. These cultured bacteria were transferred to 500 mL of fresh LB medium. When OD600 reached 0.5-0.6, 0.5 mM IPTG was added to the cultured medium to induce protein expression. The bacteria were collected after 3 hr, post-centrifugation induction. A French press was used to lyse the bacterial wall, and the protein and other components were differentiated by centrifugation. An Ni-NTA His bind resin with a His tag was applied to purify the mutant protein. Briefly, 2 ml of regenerated His resin was packed into a column. The supernatant differentiated by centrifugation was loaded into the column. The column was then washed with washing buffer to remove any contaminant proteins. The protein was eluted using elution buffer containing 500 mM imidazole. The eluent was collected and concentrated to 5 mL. The eluent was centrifuged at 12000 rpm for 10 mins, and then the supernatant was absorbed and injected with a syringe into AKTA FPLC. Before injection, the sample loop was washed with 10 mL lysis buffer. The protein was collected after passing through a size exclusion column. An SDS-PAGE gel was run to check the protein sample. All wild-type and mutant proteins were expressed and purified in this manner. Typically, the proteins were stored at −20° C., aliquoted in multiple tubes to avoid repeated freeze-thaw cycles.

The sequence of the phi29 portal protein is known and is available in Genbank (Genbank Acc. No. ACE96033). Mutant phi29 gp10 portal proteins having the following mutations were generated:
A79C;
E135C;
Q168C;
R10L, E14V, R17L and N-7Δ (mutant-b);
R10L, E14V, R17L, Q18L, R22I and N-ter-7Δ (mutant-c);
I-L added to N-terminus (mutant-d); and
R10L, E14V, R17L, N-ter-7Δ with I-L added to the N-terminus (mutant-e)

### Example 2: Pore Insertion in MinION Devices

To insert the engineered protein channel into ONT membranes, protein with 1 mg/ml concentration was diluted 1000-fold in C13 buffer (25 mM potassium phosphate, 150 mM potassium ferrocyanide, 150 mM potassium ferricyanide, pH 8). 200 μl diluted protein sample was added through the priming port of the MinION flowcell. Then a ramping voltage from +50 to +350 mV (5 mV increments; 20 s holding) was applied to assist the insertion of the protein channel. The flow cell was then flushed with 2 mL C13 buffer. An I-V curve was then run typically, ±50, ±100, ±150, ±200 mV with variable holding times (2 mins to 10 minutes holding at each voltage) to observe pore behavior over time. Analytes such as DNA or peptide (1 pM concentration) was suspended in C13 buffer and added to the flow cell to check pore functionality.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

Sequence Listing

Amino acid sequence of wild-type phi29 gp-10
SEQ ID NO: 1

| | |
|---|---|
| 1 | MARKRSNTYR SINEIQRQKR NRWFIHYLNY LQSLAYQLFE WENLPPTINP |
| 51 | SFLEKSIHQF GYVGFYKDPV ISYIACNGAL SGQRDVYNQA TVFRAASPVY |
| 101 | QKEFKLYNYR DMKEEDMGVV IYNNDMAFPT TPTLELFAAE LAELKEIISV |
| 151 | NQNAQKTPVL IRANDNNQLS LKQVYNQYEG NAPVIFAHEA LDSDSIEVFK |
| 201 | TDAPYVVDKL NAQKNAVWNE MMTFLGIKNA NLEKKERMVT DEVSSNDEQI |
| 251 | ESSGTVFLKS REEACEKINE LYGLNVKVKF RYDIVEQMRR ELQQIENVSR |
| 301 | GTSDGETNE |

1. A modified portal protein of a bacteriophage DNA packaging motor, wherein the modified portal protein is capable of direct insertion into a membrane and wherein the portal protein is modified compared to the wild type portal protein such that one or more amino acid residues on the outer surface of the portal protein is substituted by one or more other amino acid residue, and/or wherein a one or more amino acid residue is inserted on the outer surface of the portal protein so as to alter the outer surface hydrophobicity of the modified portal protein compared to the wild type portal protein.

2. The modified portal protein of claim 1, wherein at least one of the one or more amino acid residues is in the central hydrophobic belt region of the portal protein.

3. The modified portal protein of claim 1 or 2, wherein the introduction of one or more amino acid residues increases the outer surface hydrophobicity compared to the wild type portal protein.

4. The modified portal protein of any one of claims 1 to 3, wherein at least one of the one or more amino acid residues is at a position within one or two amino acids of one or more of the positions corresponding to F24, 125, L28, F60, F128, P129 and P132 of the portal protein of the Phi29 DNA packaging motor.

5. The modified portal protein of any one of claims 1 to 3, wherein at least one of the one or more amino acid residues is within about 30 amino acids of the N-terminus of the portal protein.

6. The modified portal protein of any one of the preceding claims, wherein at least one of the one or more amino acid residues is at a position corresponding to R10, E14, R17, Q18 and R22 of the portal protein of the Phi29 DNA packaging motor.

7. The modified portal protein of any one of the preceding claims, wherein at least one of the one or more amino acid residues is in the hydrophilic cis- and/or trans-layer of the portal protein.

8. The modified portal protein of claim 7, wherein at least one of the one or more amino acid residues in the cis-layer of the portal protein is at a position corresponding to Q32, Y36, F52, K55, Q59, F60, Y62, N₇₇, G78, A79, L80, S81, R84, R94, A96, S97, P98 and Q101 of the portal protein of the Phi29 DNA packaging motor and/or at least one of the one or more amino acid residues in the trans-layer of the portal protein is at a position corresponding to P129, T131, E135, Q168 of the portal protein of the Phi29 DNA packaging motor.

9. The modified portal protein of claim 8, wherein at least one of the one or more amino acid residues in the cis- or trans-layer of the portal protein is at a position corresponding to A79, E135 and/or Q168 of the portal protein of the Phi29 DNA packaging motor.

10. A modified portal protein of a bacteriophage DNA packaging motor, wherein the modified portal protein is capable of direct insertion into a membrane, wherein one or more amino acid residues is introduced on the outer surface of the portal protein, to introduce one or more binding sites on the outer side of the wing domain or in the stalk domain for a molecule that alters the hydrophobicity of the outer surface of the portal protein compared to the wild type portal protein.

11. The modified portal protein of claim 10, wherein at least one of the one or more amino acid residues introduced into the portal protein is cysteine or a non-natural amino acid.

12. The modified portal protein of claim 10 or 11, wherein at least one of the one or more amino acid residues is in the hydrophilic cis- and/or trans-layer of the portal protein.

13. The modified portal protein of claim 12, wherein at least one of the one or more amino acid residues in the cis-layer of the portal protein is at a position corresponding to Q32, Y36, F52, K55, Q59, F60, Y62, N₇₇, G78, A79, L80, S81, R84, R94, A96, S97, P98 and Q101 of the portal protein of the Phi29 DNA packaging motor and/or at least one of the one or more amino acid residues in the trans-layer of the portal protein is at a position corresponding to P129, T131, E135, Q168 of the portal protein of the Phi29 DNA packaging motor.

14. The modified portal protein of claim 13, wherein at least one of the one or more amino acid residues in the cis- or trans-layer of the portal protein is at a position corresponding to A79, E135 and/or Q168 of the portal protein of the Phi29 DNA packaging motor.

15. The modified portal protein of any one of the preceding claims, wherein the at least one amino acid is introduced by substitution and/or insertion.

16. The modified portal protein of any one of the preceding claims, wherein the portal protein is modified by the addition and/or deletion of one or more amino acid residues at the N-terminus of the portal protein.

17. The modified portal protein of any one of the preceding claims, which is a modified portal protein of a DNA packaging motor from a bacteriophage selected from the group consisting of phi29, T3, T4, T5, T7, SPP1, HK97, Lamda, G20c, P2, P3 and P22.

18. The modified portal protein of any one of the preceding claims, which is composed of identical subunits.

19. The modified portal protein of claims 10 to 18, wherein the molecule that alters the hydrophobicity of the outer surface of the portal protein compared to the wild type portal protein is a hydrophobic molecule.

20. The modified portal protein of any one of the preceding claims, wherein the hydrophobic molecule comprising porphrin, tetraphenylporphyrin, protoporphyrin IX, octaethylporphyrin, cholesterol, heme or biliverdin.

21. A subunit of the modified portal protein of any one of claims 1 to 20.

22. A membrane comprising the modified portal protein of any one of claims 1 to 20.

23. The membrane of claim 22, which is a lipid membrane or a copolymer membrane.

24. The membrane of claim 23, wherein the copolymer membrane is a diblock or triblock copolymeric membrane.

25. An array comprising two or more membranes of any one of claims 22 to 24.

26. The array of claim 25, which is adapted for insertion into a sensor device.

27. A device comprising the array of claim 25 or 26, a means for applying a voltage potential across the membranes and a means for detecting electrical charges across the membranes.

28. The device of claim 27, which further comprises a fluidics system configured to supply a sample to the membranes.

29. A method of characterising a target analyte, the method comprising contacting the membrane of any one of claims 22 to 24 with the target analyte and applying a voltage potential across the membrane such that the target analyte moves with respect to the nanopore, and taking one or more measurements as the target analyte moves with respect to the pore, thereby determining the presence, absence or one or more characteristics of the analyte.

30. The method of claim 29, wherein the measurements are electrical measurements and/or optical measurements.

31. The method of claim 29 or 30, wherein multiple target analytes are characterised.

32. The method of any one of claims 29 to 31, wherein the target analyte is a polynucleotide, protein, peptide, carbohydrate, metabolite or other chemical.

33. The method of any one of claims 29 to 32 wherein the target analyte is associated with a medical condition.
